(19) United States
(12) Patent Application Publication
(10) Pub. No.: US 2007/0117489 A1
(43) Pub. Date: May 24, 2007

(54) Nonwoven fabric for forming vehicle molded articles and use thereof

(76) Inventors: Toshiya Yamamoto, Osaka (JP); Takashi Koida, Yamaguchi (JP)

Correspondence Address: KENYON & KENYON LLP, 1500 K STREET N.W., SUITE 700, WASHINGTON, DC, 20005, US

(21) Appl. No.: 10/582,776

(22) PCT Filed: Dec. 16, 2004
(86) PCT No.: PCT/JP04/18826

§ 371 Date: Jun. 13, 2006

(30) Foreign Application Priority Data
Dec. 17, 2003 (JP) 2003-418930

(51) Int. Cl. B32B 27/12; D04H 1/46; A61F 13/15; B32B 27/32

(52) U.S. Cl. 442/396; 428/219; 428/220; 442/408; 442/364; 442/414

(57) ABSTRACT

A nonwoven fabric for forming vehicle molded articles which has a tensile stress of 0.1-20 N/5 cm at 5% elongation at 120° C. The nonwoven fabric may have a basis weight of 100 g/m² or less and an apparent density of 0.08 to 0.04 g/cm³. The fiber(s) used in the nonwoven fabric may have a fiber diameter of 1 to 100 μm. The nonwoven fabric may comprise fibers comprising a polyester material selected from the group consisting of polytrimethylene terephthalate, polybutylene terephthalate, polytetramethylene oxide glycol/polybutylene terephthalate copolymer, polyester elastomers, and amorphous polyesters.

### TECHNICAL FIELD

The present invention relates to a nonwoven fabric for forming molded articles for vehicles, which can be suitably used as a molded article or as a part thereof used in vehicles. More specifically, the invention relates to a nonwoven fabric for forming vehicle molded articles with improved cut waste recyclability.

### BACKGROUND OF THE INVENTION

Conventionally, materials having a cross-sectional configuration as shown in FIG. 1 have been used as materials for forming molded articles for vehicles such as ceiling materials, trunk materials and carpet materials. As shown in FIG. 1, the material comprises a top layer (layer A), an intermediate layer (layer B) and a bottom layer (layer C), the top layer comprising a vinyl chloride sheet, a polyester fiber woven fabric or a yarn-dyed polyester staple nonwoven fabric, the intermediate layer comprising a complex composite prepared by sandwiching a polyurethane foam between glass nonwoven fabric sheets, and the lowermost layer comprising a film-laminated nonwoven fabric.

The bottom layer of the nonwoven fabric plays a role in improving mold releasability, and preventing noise due to friction between the film, etc. of the molding material and metal parts in contact with the molding material. The bottom layer may comprise, for example, a nylon nonwoven fabric laminated with polypropylene film as described in Japanese Unexamined Patent Publication No. 2003-113569. This is because nylon nonwoven fabric is flexible and provides excellent moldability, and so fracturing during molding, flotation, etc. are unlikely to occur. Thus in view of performance, nylon nonwoven fabrics laminated with polyolefin film are often used.

For use as a molding material, it is necessary to cut the material into a shape according to the intended molded article, thereby generating a large amount of cut waste. Recycling of cut waste after molding is desired to reduce production costs and protect the global environment. However, nylon nonwoven fabric is used only in, for example, the bottom portion of molded articles, thus accounting for a low proportion of the cut waste so that recycling is difficult, and most of the waste is currently discarded.

To enhance recyclability, polyethylene terephthalate nonwoven fabrics that are used in large quantities elsewhere may be used. However, polyethylene terephthalate nonwoven fabrics have poor moldability, while fracturing of the fabric during molding causes the film portion to be exposed at the surface, so that the lowermost layer fractures due to friction with metal parts, thus generating noise. Moreover, nonwoven fabrics comprising a polyolefin material have poor heat resistance so that the abruption phenomenon of nonwoven fabric from the film, i.e., flotation of nonwoven fabric, occurs.

With regard to airbag wraps among molded articles for vehicles, since a material with particularly excellent foldability needs to be used in pillow portions of the airbag wrap, a nylon nonwoven fabric is often used. However, nylon nonwoven fabrics are expensive and have poor weatherability. Materials that are less expensive than nylon fabrics are, for example, nonwoven fabrics comprising polyolefin materials and nonwoven fabrics comprising polyester materials. Nonwoven fabrics comprising polyolefin materials have excellent foldability but poor flame retardancy and poor weather resistance. Nonwoven fabrics comprising polyester materials have excellent flame retardancy and excellent weather resistance but poor foldability. For such reasons, neither are currently used.

### DISCLOSURE OF THE INVENTION

### Problem to be Solved by the Invention

A first object of the invention is to provide a nonwoven fabric with good moldability for forming vehicle molded articles; a laminate comprising the nonwoven fabric for forming vehicle molded articles; a vehicle molded article comprising the nonwoven fabric; and a method of forming a vehicle molded article using the nonwoven fabric.

A second object of the invention is to provide a nonwoven fabric with excellent foldability for forming an airbag wrap; a laminate comprising the nonwoven fabric for forming vehicle molded articles; an airbag wrap comprising the nonwoven fabric; and a method of forming an airbag wrap using the nonwoven fabric.

A third object of the invention is to provide a nonwoven fabric with good cut waste recyclability; a laminate comprising the nonwoven fabric for forming vehicle molded articles; a vehicle molded article comprising the nonwoven fabric; and a method of forming a vehicle molded article using the nonwoven fabric.

### MEANS FOR SOLVING THE PROBLEM

The present invention provides the following nonwoven fabrics for forming vehicle molded articles, laminates for forming vehicle molded articles, vehicle molded articles, methods of forming vehicle molded articles, and uses as a material for a vehicle molded article.

Item 1. A nonwoven fabric for forming vehicle molded articles which has a tensile stress of 0.1-20 N/5 cm at 5% elongation at 120° C.

Item 2. The nonwoven fabric according to item 1 which has a basis weight of 100 g/m² or less and an apparent density of 0.08 to 0.40 g/cm³, the fibers of the nonwoven fabric having a diameter of 1 to 100 μm.

Item 3. The nonwoven fabric according to item 1 comprising fibers comprising a polyester material selected from the group consisting of polytrimethylene terephthalate, polybutylene terephthalate, polybutylene terephthalate/polytetramethylene oxide glycol copolymer, polyester elastomers, and amorphous polyesters.

Item 4. The nonwoven fabric according to item 1 obtained by hydro-entangling polyester staple fibers.

Item 5. The nonwoven fabric according to item 1 which is a polyester nonwoven fabric comprising a core/sheath composite fiber, the sheath comprising a low melting point component.

Item 6. The nonwoven fabric according to item 5 wherein the core/sheath composite fiber comprises a core comprising polybutylene terephthalate and a sheath comprising an amorphous polyester.

Item 7. The nonwoven fabric according to item 1 which is a nonwoven fabric comprising a flame-retardant polyester fiber prepared by copolymerization of a phosphate so as to have a phosphorus content of 100 to 50,000 ppm.

Item 8. The nonwoven fabric according to item 1 wherein the vehicle molded article is a vehicle ceiling material.

Item 9. The nonwoven fabric according to item 1 wherein the vehicle molded article is a vehicle trunk material.

Item 10. The nonwoven fabric according to item 1 wherein the vehicle molded article is a vehicle carpet material.

Item 11. The nonwoven fabric according to item 1 wherein the vehicle molded article is a vehicle airbag wrap.

Item 12. A laminate for forming a vehicle molded article, the laminate being obtained by laminating the nonwoven fabric of item 1 with a polyester film.

Item 13. A vehicle molded article comprising the nonwoven fabric of item 1.

Item 14. The molded article according to item 13 which is a vehicle ceiling material.

Item 15. The molded article according to item 13 which is a vehicle trunk material.

Item 16. The molded article according to item 13 which is a vehicle carpet material.

Item 17. The molded article according to item 13 which is a vehicle airbag wrap.

Item 18. A vehicle molded article comprising the laminate of item 12.

Item 19. The molded article according to item 18 which is a vehicle ceiling material.

Item 20. The molded article according to item 18 which is a vehicle trunk material.

Item 21. The molded article according to item 18 which is a vehicle carpet material.

Item 22. The molded article according to item 18 which is a vehicle airbag wrap.

Item 23. A method of forming a vehicle molded article using the nonwoven fabric of item 1.

Item 24. A method of forming a vehicle molded article using the laminate of item 12.

Item 25. Use of the nonwoven fabric of item 1 as a material for forming a vehicle molded article.

Item 26. Use of the laminate of item 12 as a material for forming a vehicle molded article.

### EFFECT OF THE INVENTION

When a molded article is formed by using the nonwoven fabric of the invention together with other materials, good moldability is obtained. More specifically, when used, for example, as the bottom layer of a vehicle molded article, such as a ceiling, trunk or carpet material, the nonwoven fabric of the invention can be molded into the desired shape because it has a low tensile stress with an elongation of 5% at 120° C. and is thus flexible.

When the nonwoven fabric of the invention is used, for example, as an airbag wrap or as a part thereof, excellent foldability, i.e., capability of being folded compactly is provided because of its high flexibility due to low tensile stress at 5% elongation at 120° C.

When the nonwoven fabric of the invention comprises polyester materials, the fabric can be profitably subjected to recycling because large amounts of polyester materials are used as vehicle molded article constituent materials. When such a nonwoven fabric of the invention is used as a laminate with a polyester film, all parts of the laminate consist of polyester, so that the cut waste can be recycled as is. Such recycling can reduce production costs and contribute to global environment improvement.

In addition, when the nonwoven fabric of the invention comprises a polyester material, it costs as little as nylon nonwoven fabrics.

### BRIEF DESCRIPTION OF THE DRAWINGS

FIG. 1 shows a cross-sectional view of a conventional material for forming vehicle molded materials. In FIG. 1, layer A is a staple fiber nonwoven fabric, layer B is an intermediate layer, and layer C is a polyester elastomer film molding base material.

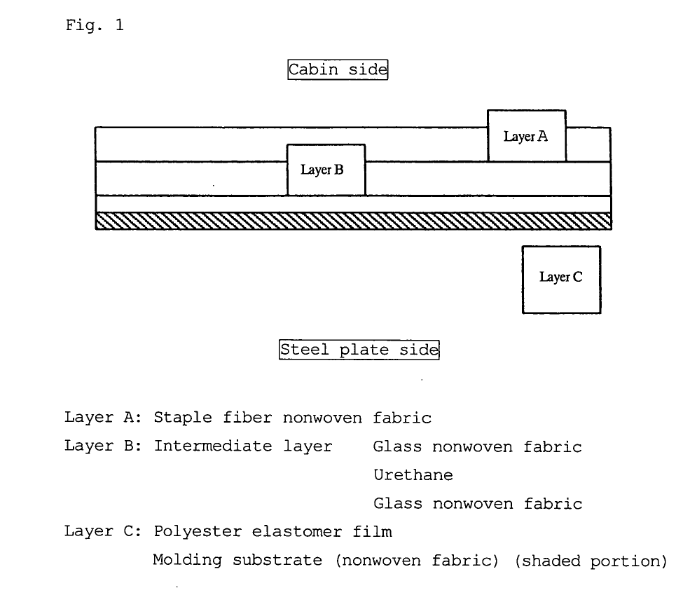

### BEST MODE FOR CARRYING OUT THE INVENTION

Characteristics of the Nonwoven Fabric

The nonwoven fabric for forming a vehicle molded article of the invention has a tensile stress of about 0.1 to about 20 N/5 cm with an elongation of 5% at 120° C., preferably about 0.3 to about 18 N/5 cm, and more preferably about 0.5 to about 17 N/5 cm. It is particularly preferable that the tensile stress in both machine and transverse directions be within the above-mentioned range.

Molded articles for vehicles are usually formed at about 120° C. Since the nonwoven fabric of the invention has a low tensile stress at 5% elongation at 120° C., good moldability can be obtained. Stated more specifically, the nonwoven fabric of the invention is easily deformable due to being within the above-mentioned range of tensile stress. Even in deep-draw molded products such as wagon bodies (vehicle ceiling material), fracturing in curved portions is unlikely to occur, so that it is unlikely that any glass urethane composite inside the molded article is exposed to the surface due to such fracturing and comes into contact with a steel plate to thereby generate noise. Furthermore, due to the above-mentioned tensile stress, the shape can be sufficiently retained during thermal molding, and problems such as exposure of inner films at the surface due to fracturing do not occur.

The nonwoven fabric of the invention preferably has a basis weight, i.e. mass per unit area, of 100 g/m² or less, more preferably 40 g/m² or less, and particularly preferably 20 g/m² or less. When the basis weight is within the above-mentioned ranges, good moldability is achieved, so that fracturing does not occur during molding. The lower limit of the basis weight is usually 5 g/m ². This is because the nonwoven fabric with such a basis weight can sufficiently function as a cushioning material when the molded article is in contact with metal.

The nonwoven fabric of the invention preferably has an apparent density of 0.08 to 0.04 g/cm³, more preferably about 0.1 to 0.4 g/cm³, and particularly preferably about 0.2 to 0.3 g/cm³. When the apparent density is within the above-mentioned ranges, good moldability can be obtained, so that fracturing does not occur even during deep-draw molding. Furthermore, the inner film does not become exposed to the surface nor come in contact with metal, etc. to thereby cause noise.

The nonwoven fabric of the invention may be a spunbond nonwoven fabric or a staple fiber nonwoven fabric. In any case, the nonwoven fabric preferably has a fiber diameter of about 1 to about 100 μm, more preferably about 1 to about 30 μm, and particularly preferably about 1 to 12 μm. When the fiber diameter is within the above-mentioned ranges, flexible sheets can be obtained and good moldability can be obtained. Even when the fiber diameter is 1 μm or less, there are no particular problems in terms of performance; however, the use of special fibers such as splittable fibers is required, thus increasing costs.

Materials of the Nonwoven Fiber

The fiber forming the nonwoven fabric preferably comprises a polyester material such as polytrimethylene terephthalate (hereinafter referred to as “PTT”); polybutylene terephthalate (hereinafter referred to as “homo-PBT”); polybutylene terephthalate (hereinafter referred to as “soft PBT”) containing an amorphous component (polytetramethylene oxide glycol (hereinafter referred to as “PTMG”), i.e., a copolymer of PTMG and soft PBT; a polyester elastomer (hereinafter referred to as “PEL”); and/or an amorphous polyester (hereinafter referred to as “soft PBT”). Such polyester materials can be used singly or in combination of two or more; a blend of homo-PBT and soft PET is particularly preferable. An elastomer as used herein refers to a polymer having a flexural modulus of 5000 kg/cm² or less.

The proportion of such material is preferably 1 wt. % or more, more preferably 10 wt. % or more, even more preferably 30 wt. % or more, and particularly preferably 50 wt. % or more. Most preferably, the nonwoven fabric consists of such fiber(s). When such fibers are used, recycling is easy. Moreover, such fibers provide good deformation follow-up capability, thus giving a nonwoven fabric that is unlikely to fracture in curved portions even in deep-draw molding.

When the nonwoven fabric constituent fiber includes a polyethylene terephthalate staple fiber, hydro-entanglement is particularly preferable, because the resulting fabric can be greatly deformed and folded compactly, thus being easy to store.

When a polyethylene terephthalate staple fiber is hydro-entangled, this is preferably performed at a low water pressure within the range of about 10 to about 50 kg/cm², more preferably about 15 to about 45 kg/cm², and even more preferably about 20 to about 40 kg/cm². When the water pressure is within the above-mentioned ranges, sufficient entanglement and reduced elongational tensile can be imparted.

The fiber used in the nonwoven fabric of the invention is preferably a fiber having a core/sheath structure with a low melting point component in the sheath. Examples of such core/sheath fibers include PET/PBT, PET/low-melting PET, PET/PE, PET/PP, PP/PE, PET/soft PBT, PET/low-melting isophthalic acid-copolymerized PET (hereinafter referred to as “CO-PET”), PET/soft PET, PET/PEL, PBT/PEL, PBT/soft PET, PBT/soft PBT, and the like. Among these, PBT/soft PET and PET/PBT are preferable, with PBT/soft PET being particularly preferable.

It is also advantageous to combine a low melting fiber as mentioned above and a high melting fiber for use as fiber mixture.

It is also advantageous to use a combination of PET/PBT, PET/PEL, PET/soft PBT, PET/soft PET, PBT/soft PBT, PBT/PEL or PBT/soft PET, and blend such materials at the chip stage for use as a blend. By blending a small amount of a low melting component, moldability or foldability as well as workability can be enhanced without incurring a large cost increase. A PBT/soft PET blended fiber is particularly preferable, since when a combination of PBT/soft PET is used, the low-melting component (typically, soft PET) selectively deposits on the surface to improve adhesion.

In the case of blended fibers, an overly large proportion of low-melting component(s) causes dripping during spinning, resulting in poor workability. The proportion of high-melting polymer to lower melting polymer(high-melting polymer/lower melting polymer) is preferably in the range of about 70/30 to about 99/1, more preferably about 80/20 to about 99/1, and even more preferably about 90/10 to about 99/1.

The nonwoven fabric of the invention is preferably comprises a flame-retardant polyester fiber prepared by copolymerization of a phosphate so as to have a phosphorus content of about 100 to about 50,000 ppm, and preferably about 500 to about 8,000 ppm. In this case, a flame-retardant fiber content of 1% or more should be sufficient. When the phosphorus content is within the above-mentioned ranges, the nonwoven fiber meets Federal Motor Vehicle Safety Standards (FMVSS). The nonwoven fabric is unlikely to have a fuzzy surface and unlikely to be caught by the mold, thus providing good workability.

Any phosphoric acid ester known as a flame retardant can be used as the phosphate. Examples of usable phosphates are phosphate compounds that react with polyester components such as dicarboxylic acids and diols to copolymerize with the polyester. Among such phosphate compounds, preferable are compounds that can introduce a phosphorus atom into a side chain and/or terminus of the polyester. Compounds that can introduce a phosphorus atom into a side chain of a polyester are particularly preferable. Examples of such phosphorus compounds are those represented by formula (1) below. 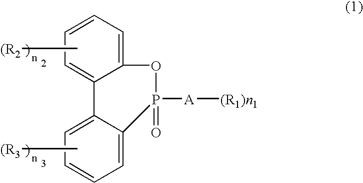
wherein R¹ is a monovalent functional group capable of ester formation; R² and R³ are same or different, and represent a halogen atom, a C₁₋₁₀ hydrocarbon group, or the same monovalent functional group as R¹; A is a divalent or trivalent organic residue; n₁ is 1 or 2; and n₂ and n₃ are same or different, and represent an integer from 0 to 4.

Specific examples of compounds of formula (1) are those represented by the following formulae (a) to (z) and (α) and (β). 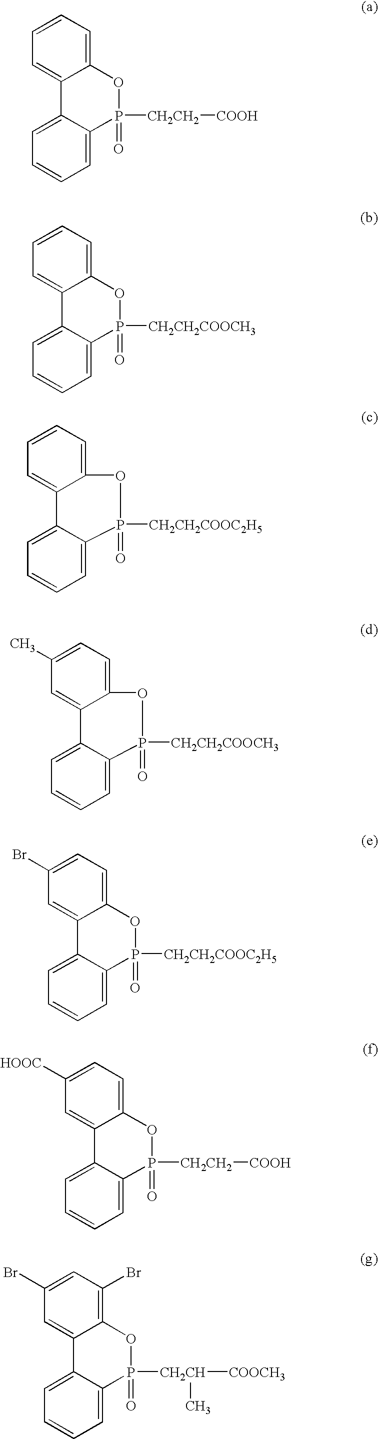 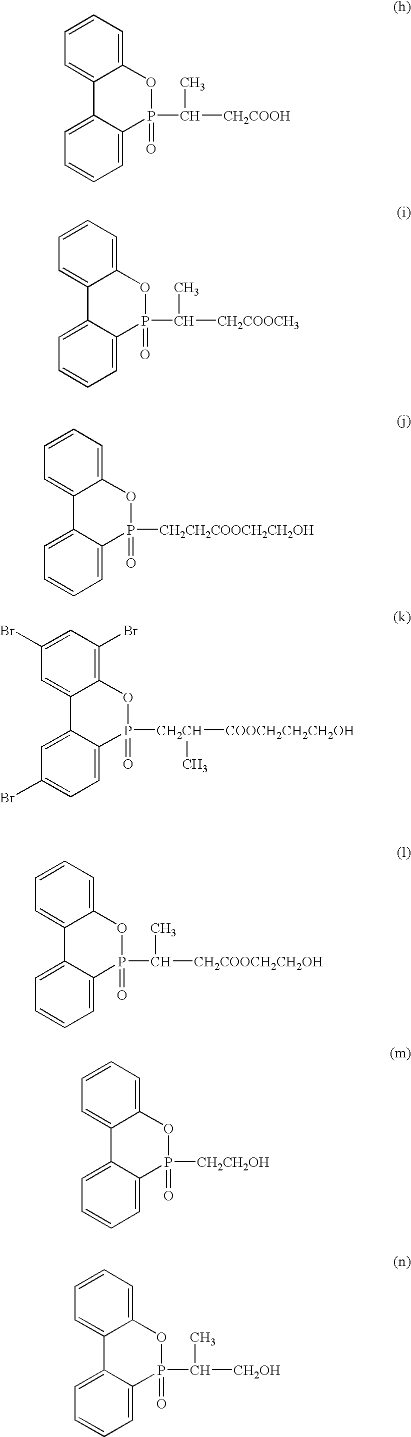 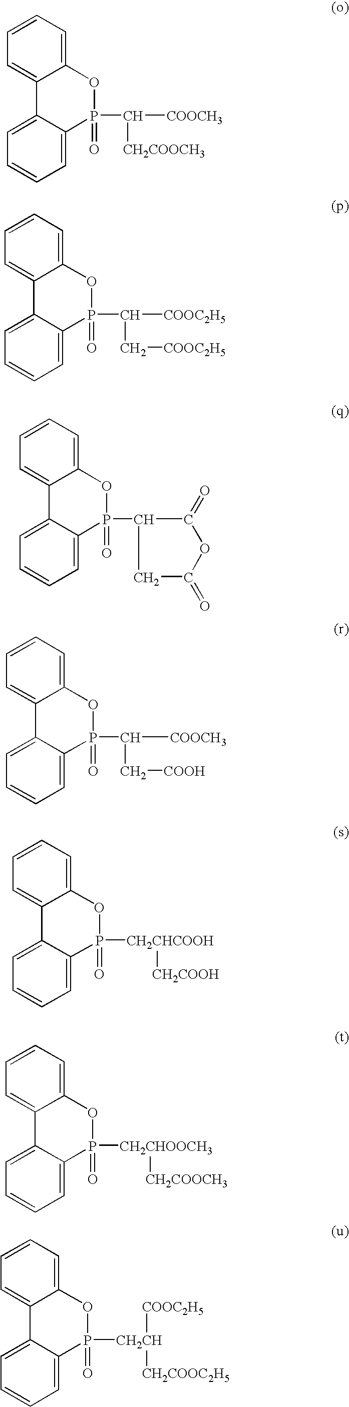 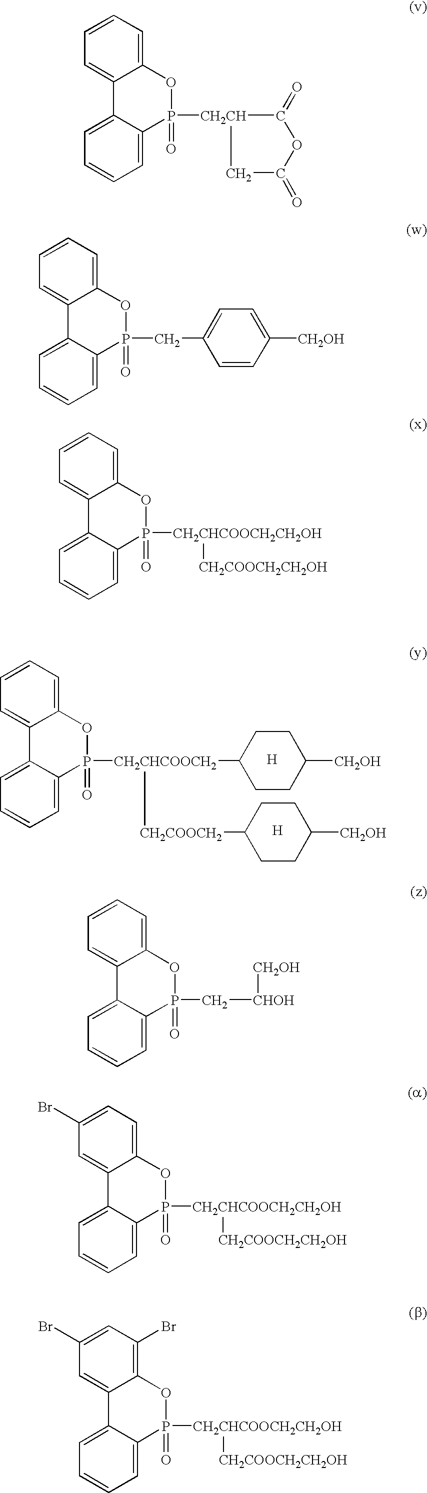
(2-carboxyethyl)phenylphosphinic acid, i.e. 2-carboxyethyl phenylphophinate is also preferable as a phosphate compound of the invention.
Use of the Nonwoven Fabric

The nonwoven fabric of the invention is used to form molded articles used in vehicles. Examples of vehicles include trains, automobiles, and the like. Examples of molded articles include ceiling materials, trunk materials, carpet materials, airbag wraps and the like. Since the nonwoven fabric of the invention has good foldability, it is particularly suitable for use as a nonwoven fabric for forming a vehicle airbag wrap. When used for ceiling materials, trunk materials, carpet materials, airbag wraps or the like, the nonwoven fabric is used to constitute at least part of the molded article and is usually used in the lowermost or uppermost layer. When used as an airbag wrap, the nonwoven fabric of the invention may constitute the entire wrap, or may be just a part thereof. In other words, vehicle ceiling materials, trunk materials, carpet materials, and airbag wraps of the invention comprise the above-mentioned nonwoven fabric of the invention.

In particular, airbag wraps are provided with improved foldability by use of the nonwoven fabric of the invention. When the nonwoven fabric constituent fiber(s) comprise a polyester fiber, the resulting nonwoven fabric is provided with excellent weather resistance, excellent flame retardancy, and low-cost, and in particular, has lower-cost and better weather resistance than nylon nonwoven fabrics.

Laminate for Forming Vehicle Molded Articles

The laminate for forming vehicle molded articles of the invention is a laminate of the above-mentioned nonwoven fabric of the invention with a film comprising a polyester material. Any known polyester material hitherto used as a film material in vehicle ceiling materials, vehicle trunk materials, vehicle carpet materials, airbag wraps, etc. may be used as the polyester material. Examples of such polyester materials include PET and PBT. PBT is particularly preferable because it has high elongation and good flexibility and is also capable of deforming in accordance with stretching of the nonwoven fabric of the invention. The thickness of the polyester film may usually be about 8 to about 40 μm. Although the number of the sheets of the nonwoven fabric and the film used is not particularly limited, a laminate of one sheet of the nonwoven fiber with one sheet of polyester film is usual.

Such a laminate can also be used to mold, for example, vehicle ceiling materials, vehicle trunk materials, vehicle carpet materials and vehicle airbag wraps. The laminate of the invention is usually used to constitute part of a vehicle ceiling material, vehicle trunk material or vehicle carpet material, or to constitute part or all of a vehicle airbag wrap.

Method of Forming Vehicle Molded Articles

The method of forming a vehicle molded article of the invention is a method of forming a molded article using the above-mentioned nonwoven fabric or laminate of the invention.

More specifically, the method may comprise placing the nonwoven fabric or laminate of the invention in contact with a mold and laying a polyurethane foam material thereon to be molded in such a manner that the nonwoven fabric or laminate is in contact with the polyurethane foam. When a laminate is used, it may usually be molded in such a manner that the nonwoven fabric is outermost.

Airbag wraps may be formed, for example, by sewing, or cutting and pasting the nonwoven fabric or laminate of the invention into the intended shape of the airbag wrap.

### EXAMPLES

The present invention is described below in more detail with reference to Examples. The invention, however, is not limited thereto.

The nonwoven fabrics and molded articles obtained in the Examples and Comparative Examples were evaluated according to the following:

(1) Basis Weight (g/m²)

Ten samples 5 cm wide and 20 cm long were obtained from a test fabric by cutting 5 strips each spaced 5 cm apart in the transverse direction and 5 strips each spaced 5 cm apart in the longitudinal direction. The mean basis weight thereof was calculated.

(2) Thickness (mm)

Five samples were obtained by cutting strips each spaced 20 cm apart in the widthwise direction. The thickness of the samples was measured using a thickness gauge (with a load of 25 g/cm² being applied to a load area of 4 cm²).

(3) Apparent Density (g/cm³)

Apparent density was calculated by the following formula: Apparent density=Basis weight (g/m²)/(Thickness (cm)×100×100)

(4) Tensile Stress at High Temperature (ST5) (N/5 cm)

Evaluation was made using a thermal tensile tester (model: RTC-1250, manufactured by Orientec Co.) RT at a head speed of 10 cm/min with a chuck-to-chuck distance of 20 cm. After being heated at 65° C. for 1 minute and then at 120° C. for 1 minute, the samples were elongated at 120° C. The tensile stress at 5% elongation was determined (ST5).

(5) Spinning Operability

The occurrence of thread dripping during spinning was visually inspected and evaluated based on the following criteria:

◯: Thread dripping hardly occurred.

X: Thread dripping occurred frequently.

(6) Moldability

The condition of a nonwoven fabric sheet in curved portions of the molded article and pillow portions was visually inspected, and evaluated according to the following criteria:

◯: Neither nonwoven fabric sheet fracturing in curved portions of the mold nor floating in pillow portions due to peeling of the nonwoven fabric from the film occurred.

Δ: Nonwoven fabric sheet fracturing did not occur in curved portions of the mold, but flotation occurred in pillow portions due to peeling of the nonwoven fabric from the PET film.

X: Nonwoven fabric sheet fracturing occurred in curved portions of the mold.

(7) Surface Fuzzing

Sheet materials obtained in the Examples and Comparative Examples below were tested for workability and evaluated according to the following criteria:

◯: After molding, able to be released from the mold smoothly without edges of the substrate being caught in the mold.

X: After molding, edges of the substrate were caught, thus being difficult to release from the mold.

(8) Noise due to friction with a steel plate

Automobiles were produced incorporating the sheet materials obtained in the Examples and Comparative Examples, and evaluated for noise heard by someone seated in the cars.

◯: Substantially no noise was heard by someone seated in the cars.

X: Noise was always heard by someone seated in the cars.

### Examples 1-3

A randomly looped web of 1.5 dtex polyester fiber with a basis weight as shown in Table 1 below was formed by spunbonding using materials as shown in Table 1 and prepared by copolymerization with a phosphorus-containing compound, i.e., (2-carboxyethyl) phenylphosphinic acid in an amount as shown in Table 1. The web was calendered by pressing between rollers heated at 210° C. to provide a molding substrate. A 30 μm polyethylene film was laminated on the molding substrate thus obtained. The resulting laminate was molded together with a polyurethane foam material at 120° C. and used as a lowermost layer to produce an automobile ceiling material. The laminate cut waste of the lowermost layer was recycled as a recycled polyester fiber material felt.

The laminate of the above-mentioned nonwoven fabric with the above-mentioned film was used also as a pillow portion of an airbag wrap for use in automobiles. The wrap material showed a good foldability for storage.

### Example 4

A randomly looped web of 1.5 dtex mixed fiber of polyethylene terephthalate and a polyester elastomer with a basis weight of 20 g/m² was formed by spunbonding and prepared by copolymerization with a phosphorus-containing compound, i.e., (2-carboxyethyl)phenylphosphinic acid in the amount shown in Table 1. The web was calendered by pressing between rollers heated at 235° C. to provide a molding substrate.

A 30 μm polyethylene film was laminated on the molding substrate thus obtained. The resulting laminate was molded together with a polyurethane foam material and used as a lowermost layer to produce an automobile ceiling material. The laminate cut waste of the lowermost layer was recycled as a rough polyester fiber material felt.

### Examples 5-9

A randomly looped web of 1.5 dtex polyester fiber blend comprising two kinds of materials shown in Table 1 below with a basis weight of 20 g/m² was formed by spunbonding and prepared by copolymerization with a phosphorus-containing compound, i.e., (2-carboxyethyl)phenylphosphinic acid in an amount as shown in Table 1. The web was calendered by pressing between rollers heated at 210° C. to provide a molding substrate.

An aromatic polyester copolymer with a melting point of 178° C. and an intrinsic viscosity of 0.780 was obtained by charging 100 parts of terephthalic acid, 40 parts of ethylene glycol and 15 parts of neopentyl glycol together with a small amount of a catalyst and allowing an ester-coupling interchange polymerization to proceed by an usual method, followed by pelletization. The aromatic polyester copolymer was used as soft PET.

A 30 μm polyethylene film was laminated on the molding substrate thus obtained. The resulting laminate was molded together with a polyurethane foam material at 120° C. and used as a lowermost layer to produce an automobile ceiling material. The laminate cut waste of the lowermost layer was recycled as a rough polyester fiber material felt.

### Examples 10 and 11

A randomly looped web of 1.5 dtex core/sheath polyester fibers with a basis weight of 20 g/m² was formed by spunbonding using material A shown in Table 1 below as the core component and material B shown in Table 1 as the sheath component and prepared by copolymerization with a phosphorus-containing compound, i.e., (2-carboxyethyl)phenylphosphinic acid in the amount shown in Table 1. The web was calendered by pressing between rollers heated at 210° C. to provide a molding substrate.

A 30 μm polyethylene film was laminated on the molding substrate thus obtained. The resulting laminate was molded together with a polyurethane foam material at 120° C. and used as a lowermost layer to produce an automobile ceiling material. The laminate cut waste of the lowermost layer was recycled as a rough polyester fiber material felt.

### Example 12

Polyester staple fibers with a staple length of 51 mm and a fineness of 2.0 dtex prepared by copolymerization with a phosphorus-containing compound, i.e., (2-carboxyethyl)phenylphosphinic acid in the amount shown in Table 1 were hydro-entangled at a water pressure of 40 kg/cm² and dried by air-through method to provide a staple fiber nonwoven fabric with a basis weight of 25 g/m² and a thickness of 0.25 mm.

A 30 μm polyethylene film was laminated on the obtained nonwoven fabric. The resulting laminate was molded together with a polyurethane foam material at 120° C. and used as a lowermost layer to produce an automobile ceiling material. The laminate cut waste of the lowermost layer was recycled as a rough polyester fiber material felt.

### Comparative Example 1

A randomly looped web of 2.2 dtex polyethylene terephthalate fiber with a basis weight of 20 g/m² was formed by spunbonding and prepared by copolymerization with a phosphorus-containing compound, i.e., (2-carboxyethyl)phenylphosphinic acid in the amount shown in Table 1. The web was calendered by pressing between rollers heated at 242° C. to provide a molding substrate.

A 30 μm polyethylene film was laminated on the molding substrate thus obtained. The resulting laminate was molded together with a polyurethane foam material in order to be used as a lowermost layer in an automobile ceiling material. However, the sheet fractured in curved portions of the mold during molding.

### Comparative Example 2

A randomly looped web of 2.2 dtex polypropylene (PP) fiber with a basis weight of 20 g/m² was formed by spunbonding. The web was calendered by pressing between rollers heated at 130° C. to provide a molding substrate.

A 30 μm polyethylene film was laminated on the molding substrate thus obtained. The resulting laminate was molded together with a polyurethane foam material at 120° C. and used as a lowermost layer to produce an automobile ceiling material. After molding, peeling of the PP nonwoven fabric from the film occurred in pillow portions. Furthermore, when the molded article was released from the mold, the surface of the nonwoven fabric was caught and fractured.

Since only small amounts of PP nonwoven fabric are used in automobiles, it was impossible to recycle the cut waste to produce a recycled polyester fiber material felt, and all the waste was discarded. The PP nonwoven fabric was tested for flame retardancy according to a FMVSS method, i.e., a test method for vehicle flame retardancy, but did not pass the test.

### Comparative Example 3

A randomly looped web of 2.2 dtex nylon fiber with a basis weight of 20 g/m² was formed by spunbonding and prepared by copolymerization with a phosphorus-containing compound, i.e., (2-carboxyethyl)phenylphosphinic acid in the amount shown in Table 1. The web was calendered by pressing between rollers heated at 210° C. to provide a molding substrate.

A 30 μm polyethylene film was laminated on the molding substrate thus obtained. The resulting laminate was molded together with a polyurethane foam material at 120° C. and used as a lowermost layer to produce an automobile ceiling material.

When the molded article was released from the mold, the surface portion of the nonwoven fabric was caught and fractured. Since only small amounts of nylon nonwoven fabric are used in automobiles, it was impossible to recycle the cut waste to produce a recycled polyester fiber material felt, and all the waste was discarded.

### Comparative Example 4

A randomly looped web of 2.2 dtex polyethylene terephthalate fiber with a basis weight of 131 g/m² was formed by spunbonding and prepared by copolymerization with a phosphorus-containing compound, i.e., (2-carboxyethyl)phenylphosphinic acid in an amount shown in Table 1. The web was calendered by pressing between rollers heated at 252° C. to provide a molding substrate.

A 30 μm polyethylene film was laminated on the molding substrate thus obtained. The resulting laminate was molded together with a polyurethane foam material and used as a lowermost layer to produce an automobile ceiling material. The sheet fractured in curved portions of the mold during molding.

### Comparative Example 5

A randomly looped web of 2.2 dtex bicomponent fibers with a basis weight of 20 g/m² and a core/sheath weight ratio of 40:60 (core: polyethylene terephthalate (melting point: 260° C.)/sheath: polyethylene (melting point: 130° C.). The web was calendered by pressing between rollers heated at 110° C. to provide a molding substrate.

A 50 μm polyester elastomer film was laminated on the molding substrate thus obtained. The resulting laminate was molded together with a polyurethane foam material at 120° C. and used as a lowermost layer to produce an automobile ceiling material.

Since only small amounts of polyethylene nonwoven fabric are used in automobiles, it was impossible to recycle the cut waste to produce a recycled polyester fiber material felt, and all the waste was discarded.

The above nonwoven fabric was tested for flame retardancy according to a FMVSS method, i.e., a test method for vehicle flame retardancy, but did not pass the test.

### Comparative Example 6

An attempt was made to form a nonwoven web of 1.5 dtex polyester resin blended fiber (with a polyethylene terephthalate: soft polybutylene terephthalate weight ratio of 20:80) by spunbonding. However, dripping frequently occurred during spinning, thus failing to form a nonwoven fabric sheet.

TABLE 1

| | Mono- | | | | | | | | |
|---|---|---|---|---|---|---|---|---|---|
| | component, | | | | | | | Tensile stress at | |
| | Blend, | | | | | | | 5% elongation at | |
| | Core/sheath, | | | | Fiber | Basis | | 120° C. | |
| | or Fiber | | | Ratio | diameter | weight | Thickness | Longitudinal | Transverse |
| | mixture | Material A | Material B | (%) | μm | (g/m²) | mm | (N/5 cm) | (N/5 cm) |
| Example 1 | Mono- | PBT | — | 100/0 | 11.8 | 20 | 0.14 | 6.7 | 1.6 |
| | component | | | | | | | | |
| Example 2 | Mono- | PBT | — | 100/0 | 11.8 | 50 | 0.25 | 16.8 | 4.0 |
| | component | | | | | | | | |
| Example 3 | Mono- | PTT | — | 100/0 | 11.8 | 20 | 0.14 | 7.0 | 1.4 |
| | component | | | | | | | | |
| Example 4 | Fiber | PET | PEL | 85/15 | 11.8 | 20 | 0.14 | 8.0 | 2.0 |
| | mixture | | | | | | | | |
| Example 5 | Blend | PET | Soft | 85/15 | 11.8 | 20 | 0.14 | 9.0 | 3.0 |
| | | | PET | | | | | | |
| Example 6 | Blend | PBT | PEL | 85/15 | 11.8 | 20 | 0.14 | 5.0 | 0.5 |
| Example 7 | Blend | PET | Soft | 85/15 | 11.8 | 20 | 0.14 | 7.5 | 2.6 |
| | | | PBT | | | | | | |
| Example 8 | Blend | PBT | Soft | 87/13 | 11.8 | 25 | 0.15 | 18.0 | 5.0 |
| | | | PET | | | | | | |
| Example 9 | Blend | PBT | Soft | 93/7 | 11.8 | 20 | 0.14 | 16.0 | 4.2 |
| | | | PET | | | | | | |
| Example | Core/sheath | PET | CO- | 40/60 | 11.8 | 20 | 0.14 | 5.2 | 0.8 |
| 10 | | | PET | | | | | | |
| Example | Core/sheath | PBT | PEL | 40/60 | 11.8 | 20 | 0.14 | 5.0 | 0.5 |
| 11 | | | | | | | | | |
| Example | Mono- | PET | — | 100/0 | 15 | 27 | 0.27 | 3.7 | 0.2 |
| 12 | component | | | | | | | | |
| Comp. Ex. 1 | Mono- | PET | — | 100/0 | 11.8 | 20 | 0.14 | 21.9 | 9.1 |
| | component | | | | | | | | |
| Comp. Ex. 2 | Mono- | PP | — | 100/0 | 11.8 | 20 | 0.14 | 7.0 | 2.0 |
| | component | | | | | | | | |
| Comp. Ex. 3 | Mono- | NY | — | 100/0 | 11.8 | 20 | 0.14 | 5.4 | 1.2 |
| | component | | | | | | | | |
| Comp. Ex. 4 | Mono- | PET | — | 100/0 | 14.1 | 131 | 0.46 | 141.7 | 59.2 |
| | component | | | | | | | | |
| Comp. Ex. 5 | Core/sheath | PET | PE | 40/60 | 14.1 | 20 | 0.14 | 5.0 | 0.9 |
| Comp. Ex. 6 | Blend | PET | Soft | 20/80 | 14.1 | — | — | — | — |
| | | | PBT | | | | | | |

| | | | | Evaluation on | | | |
|---|---|---|---|---|---|---|---|
| | | | | molded article | | | |
| | | Flame retardancy | | | | Noise by | |
| | | Phosphorus | | | | friction | |
| | | content of | | | | with a | |
| | Spinning | the substrate | | | Surface | steel | |
| | performance | (ppm) | FMVSS | Moldability | fuzzing | plate | Recyclability |
| Example 1 | ◯ | 300 | ◯ | ◯ | ◯ | ◯ | ◯ |
| Example 2 | ◯ | 300 | ◯ | ◯ | ◯ | ◯ | ◯ |
| Example 3 | ◯ | 500 | ◯ | ◯ | ◯ | ◯ | ◯ |
| Example 4 | ◯ | 300 | ◯ | ◯ | ◯ | ◯ | ◯ |
| Example 5 | ◯ | 500 | ◯ | ◯ | ◯ | ◯ | ◯ |
| Example 6 | ◯ | 300 | ◯ | ◯ | ◯ | ◯ | ◯ |
| Example 7 | ◯ | 300 | ◯ | ◯ | ◯ | ◯ | ◯ |
| Example 8 | ◯ | 300 | ◯ | ◯ | ◯ | ◯ | ◯ |
| Example 9 | ◯ | 300 | ◯ | ◯ | ◯ | ◯ | ◯ |
| Example | ◯ | 500 | ◯ | ◯ | ◯ | ◯ | ◯ |
| 10 | | | | | | | |
| Example | ◯ | 500 | ◯ | ◯ | ◯ | ◯ | ◯ |
| 11 | | | | | | | |
| Example | ◯ | 300 | ◯ | ◯ | ◯ | ◯ | ◯ |
| 12 | | | | | | | |
| Comp. Ex. 1 | ◯ | 100 | ◯ | X | ◯ | X | ◯ |
| Comp. Ex. 2 | ◯ | — | X | ◯ | X | ◯ | X |
| Comp. Ex. 3 | ◯ | 500 | ◯ | ◯ | X | ◯ | X |
| Comp. Ex. 4 | ◯ | 500 | ◯ | X | ◯ | X | ◯ |
| Comp. Ex. 5 | ◯ | — | X | ◯ | ◯ | ◯ | X |
| Comp. Ex. 6 | X | — | — | — | — | — | — |

In the core/sheath fibers, the core is of material A and the sheath is of material B.

In Table 1, in the FMVSS flame retardancy test column, ◯ indicates “passed” and X indicates “failed”.

### INDUSTRIAL APPLICABILITY

The nonwoven fabric and laminate of the invention have excellent moldability. When the fabric or laminate comprises a polyester material, recycling is easy. Therefore, the nonwoven fabric and laminate of the invention can be suitably used as a constituent material of vehicle molded articles such as vehicle ceiling materials, trunk materials, carpet materials, and airbag wraps.

1. A nonwoven fabric for forming vehicle molded articles which has a tensile stress of 0.1-20 N/5 cm at 5% elongation at 120° C.

2. The nonwoven fabric according to claim 1 which has a basis weight of 100 g/m² or less and an apparent density of 0.08 to 0.04 g/cm³, the fibers of the nonwoven fabric having a diameter of 1 to 100 μm.

3. The nonwoven fabric according to claim 1 comprising fibers comprising a polyester material selected from the group consisting of polytrimethylene terephthalate, polybutylene terephthalate, polybutylene terephthalate/polytetramethylene oxide glycol copolymer, polyester elastomers, and amorphous polyesters.

4. The nonwoven fabric according to claim 1 obtained by hydro-entangling polyester staple fibers.

5. The nonwoven fabric according to claim 1 which is a polyester nonwoven fabric comprising a core/sheath composite fiber, the sheath comprising a low melting point component.

6. The nonwoven fabric according to claim 5 wherein the core/sheath composite fiber comprises a core comprising polybutylene terephthalate and a sheath comprising an amorphous polyester.

7. The nonwoven fabric according to claim 1 which is a nonwoven fabric comprising a flame-retardant polyester fiber prepared by copolymerization of a phosphate so as to have a phosphorus content of 100 to 50,000 ppm.

8. The nonwoven fabric according to claim 1 wherein the vehicle molded article is a vehicle ceiling material.

9. The nonwoven fabric according to claim 1 wherein the vehicle molded article is a vehicle trunk material.

10. The nonwoven fabric according to claim 1 wherein the vehicle molded article is a vehicle carpet material.

11. The nonwoven fabric according to item 1 wherein the vehicle molded article is a vehicle airbag wrap.

12. A laminate for forming a vehicle molded article, the laminate being obtained by laminating the nonwoven fabric of item 1 with a polyester film.

13. A vehicle molded article comprising the nonwoven fabric of claim 1.

14. The molded article according to claim 13 which is a vehicle ceiling material.

15. The molded article according to claim 13 which is a vehicle trunk material.

16. The molded article according to claim 13 which is a vehicle carpet material.

17. The molded article according to claim 13 which is a vehicle airbag wrap.

18. A vehicle molded article comprising the laminate of claim 12.

19. The molded article according to claim 18 which is a vehicle ceiling material.

20. The molded article according to claim 18 which is a vehicle trunk material.

21. The molded article according to claim 18 which is a vehicle carpet material.

22. The molded article according to claim 18 which is a vehicle airbag wrap.

23. A method of forming a vehicle molded article using the nonwoven fabric of claim 1.

24. A method of forming a vehicle molded article using the laminate of claim 12.

25. Use of the nonwoven fabric of claim 1 as a material for forming a vehicle molded article.

26. Use of the laminate of claim 12 as a material for forming a vehicle molded article.
